(19) United States
(12) Patent Application Publication
(10) Pub. No.: US 2024/0302251 A1
(43) Pub. Date: Sep. 12, 2024

(54) MONITORING DEVICE AND METHOD FOR MONITORING THE QUALITY OF A GAS ATMOSPHERE

(71) Applicant: EBM-PAPST MULFINGEN GMBH & CO. KG, Mulfingen (DE)

(72) Inventors: Thomas SAUER, Mergentheim (DE); Jan GEHRINGER, Rothenburg o.d. Tauber (DE)

(21) Appl. No.: 18/573,759

(22) PCT Filed: May 30, 2022
(86) PCT No.: PCT/EP2022/064606

§ 371 Date: Dec. 22, 2023

(30) Foreign Application Priority Data
Jun. 22, 2021 (DE) 10 2021 116 154.1

(51) Int. Cl. G01N 1/22; G01F 1/34; G01N 1/24; G01N 33/00

(52) U.S. Cl. CPC G01N 1/2273; G01F 1/34; G01N 1/24; G01N 33/0073; G01N 2001/245

(57) ABSTRACT

A monitoring device and to a method for monitoring the quality of a gas atmosphere. The monitoring device has a ventilator which is designed to produce a gas stream having a variable volumetric flow. A gas sensor is arranged at a sensor position on a stationary part of the ventilator and provides a measurement value for a control device. The control device is designed to determine a corrected monitoring value of the volumetric flow based on the measurement value and provided correction data.

This application is a National Stage of PCT Application No. PCT/EP2022/064606 filed on May 30, 2022, which claims priority to German Patent Application No. 10 2021 116 154.1 filed on Jun. 22, 2021, the contents each of which are incorporated herein by reference thereto.

### TECHNICAL FIELD

The present disclosure refers to a monitoring device and a method for monitoring the quality of a gas atmosphere. In addition, the present disclosure refers to a method for setup of the monitoring device, particularly prior to or during start-up.

### BACKGROUND

A sensor device for monitoring the quality of a gas atmosphere is described in US 2021/0140868 A1. With this sensor device the particle concentration in an aerosol is measured. The particle concentration is detected based on the electrical load of the particles flowing through the sensor. An economic and reliable measurement shall be carried out also of ultrafine particles smaller than 0.1 μm. For this purpose, a response function for determination of the particle concentration is created that is independent from the volumetric flow rate of the aerosol flowing into the sensor device.

EP 2 142 857 B1 describes a control device and a method for indicating of the room air quality. The volumetric flow rate of the supplied air can either be temperature controlled or air quality controlled in a closed loop manner. During the first installation of the control device or during the operation the logarithmic measurement scale for the air quality of the air quality sensor can be shifted depending on the actual room temperature in order to calibrate the air quality sensor.

The precise determination of the quality of a gas atmosphere is elaborate. In many known gas sensors for determination of the gas quality the measurement value depends on the volumetric flow rate through the gas sensor. For this reason, it is regularly not possible to measure directly on a fan of a room air system. Typically, a small fan is assigned to the gas sensor that creates a constant volumetric flow rate of a measurement flow that flows through the gas sensor, independent from the operation of the (main) fan of the room air system. The measurement accuracy that can be achieved depends in this case on the measurement flow remaining unaffected by the gas flow generated by the fan of a room air system.

### BRIEF SUMMARY

Therefore, it can be considered as object of the present disclosure to simplify the monitoring of the quality of a gas atmosphere and particularly allow a simple integration into a room air system.

Disclosed is a monitoring device configured for monitoring the quality of a gas atmosphere, including: a fan configured to create a gas flow having a variable volumetric flow rate and comprising a fan rotor and an electric motor, wherein the fan rotor is rotatingly supported around a rotation axis on a fan stator, a gas sensor arranged on the fan stator or on the electric motor and being configured to produce a measurement value characterizing the quality of the gas of the gas atmosphere, a control device, which is communicatively connected to the gas sensor and which determines a volumetric flow rate corrected monitoring value based on the measurement value, the current volumetric flow rate and correction data.

Also disclosed is a method for set-up of a monitoring device for monitoring the quality of a gas atmosphere, wherein the monitoring device comprises a fan for producing a gas flow having a variable volumetric flow rate having a fan rotor and an electric motor, wherein the fan rotor is rotatingly supported around a rotation axis on a fan stator, a gas sensor arranged on the fan stator or an the electric motor and a control device communicatively connected to the gas sensor, wherein the method comprises the following steps: determining a reference value for the quality of the gas of the gas atmosphere, operating the fan so that the volumetric flow rate varies from a minimum volumetric flow rate to a maximum volumetric flow rate and creating a calibration measurement value at different volumetric flow rates respectively, which characterize the quality of the gas of the gas atmosphere, storing the reference value and the value pairs consisting of one calibration measurement value and the assigned volumetric flow rate respectively as correction data or determining one correction value respectively from the reference value and one of the calibration measurement values and storing the value pairs consisting of one correction value and the assigned volumetric flow rate respectively as correction data.

Also disclosed is a method for monitoring the quality of a gas atmosphere using a monitoring device, including a fan for producing a gas flow having a variable volumetric flow rate having a fan rotor and an electric motor, wherein the fan rotor is rotatingly supported around a rotation axis on a fan stator, a gas sensor arranged on the fan stator or the electric motor and a control device communicatively connected to the gas sensor, wherein the method comprises the following steps: detection of a measurement value that characterizes the quality of the gas of the gas atmosphere by means of the gas sensor, determining a volumetric flow rate corrected monitoring value based on the measurement value, the current volumetric flow rate and predefined correction data.

The monitoring device according to the present disclosure is configured to monitor the quality of a gas atmosphere. The gas atmosphere is particularly an air atmosphere. For example, for determination of the quality of the gas atmosphere the concentration of dust particles and/or the concentration of one, multiple or all volatile organic compounds (VOC) can be determined. For example, the concentration of the dust particles having a predefined size (e.g. PM 2.5) or a TVOC value (Total Volatile Organic Compounds) can be determined.

Additionally or alternatively, also other values can be measured, which describe the condition and/or the quality of the gas atmosphere, e.g. the temperature, the humidity, the concentration of at least one chemical content of the gas atmosphere, such as the concentration of carbon dioxide (CO₂) or nitrogen oxides (NOₓ).

The monitoring device has a fan, which is configured for creation of a gas flow. The volumetric flow rate of the gas flow is thereby modifiable depending on the operating condition of the fan. The fan is controlled by means of a control device in open loop or closed loop manner, wherein the volumetric flow rate or a differential pressure between the suction side and the pressure side of the fan can be controlled in open loop or closed loop manner, for example. The fan and the control device can be part of a room air system, e.g. for air supply and/or air discharge (e.g. fresh air and/or warm air and/or cold air, . . . ) in or out of a room or multiple rooms in a building.

The fan has an electric motor and a fan rotor that is rotatably supported around a rotation axis on a fan stator. The electric motor is drivingly connected with the fan rotor and configured to drive the fan rotor rotatingly around the rotation axis.

The monitoring device has in addition a gas sensor that is communicatively connected with the control device. The gas sensor provides a measurement value to the control device, which indicates the quality of the gas of the gas atmosphere, i.e. for example the concentration of dust particles and/or volatile organic contents in the gas atmosphere. In addition, at least one additional sensor can be provided in order to measure an additional parameter (e.g. at least one physical parameter) and to provide it to the control device, such as the temperature and/or the humidity and/or the pressure of the gas atmosphere.

The gas sensor is arranged on the fan stator or on the electric motor or a motor housing of the electric motor. The gas sensor is preferably arranged outside of the main flow path of the gas flow. Particularly, the gas sensor is arranged at a sensor position that has been determined by simulation and/or measurement over the working range or operating range of the fan and in which the volumetric flow rate changes as little as possible, if the fan creates the gas flow with a variable volumetric flow rate. For example, the sensor position can be located at a surface facing downstream, so-to-speak in the slipstream of the gas flow. The sensor position can be determined by means of measurement and/or simulation.

The control device is configured to determine a monitoring value, which is volumetric flow rate corrected and at least substantially volumetric flow rate independent, based on the measurement value of the gas sensor, the current volumetric flow rate of the fan and correction data stored or provided otherwise. For example, correction data can be determined by means of simulation and/or measurement prior to or during start-up of the fan or the room air system comprising the fan and can be stored in a non-volatile memory of the control device and/or in a non-volatile external memory with which the control device is communicatively connected (e.g. cloud memory). The correction data can be provided in the form of a table, a curve or a function and can describe the correlation between the measurement value, the current volumetric flow rate and the volumetric flow rate corrected monitoring value.

In doing so it is possible to use fans having a variable volumetric flow rate for an accurate determination of the quality of a gas atmosphere. The gas sensor can thus form a unit with the fan and does not have to be arranged separately as far away from the fan as possible. The installation of the fan or a room air system at an installation site, e.g. inside a building, is thereby remarkably simplified.

It is advantageous, if the monitoring device comprises in addition a volumetric flow rate sensor for measurement of the respectively current value of the volumetric flow rate. The volumetric flow rate sensor is preferably arranged inside the main flow path of the gas flow with distance to the gas sensor. The volumetric flow rate sensor is communicatively connected to the control device and provides the respectively currently determined value of the volumetric flow rate to the control device.

It can also be advantageous to determine a current value for the volumetric flow rate without the use of a volumetric flow rate sensor by means of the control device. For this purpose, at least one operation parameter of the fan and/or the electric motor can be provided to the control device, e.g. the rotational speed of the fan and the torque of the electric motor or one or more additional operation parameters that characterize the rotational speed of the fan and the torque of the electric motor. For example, the torque of the electric motor can be characterized by means of the motor current. The rotational speed of the fan can be described by means of the predefined setpoint rotational speed of the control device or can be detected by means of a rotational speed sensor.

The control device can be configured to use the differential pressure or the volumetric flow rate as command variable for the open loop or closed loop control.

In a preferred embodiment the monitoring device comprises in addition a differential pressure sensor arrangement for measurement of the differential pressure between a suction side and a pressure side of the fan. The differential pressure sensor arrangement is communicatively connected to the control device. The differential pressure sensor arrangement can be omitted in another embodiment if the control device is configured to control the differential pressure.

As explained at the beginning, the gas sensor is particularly arranged outside of the main gas flow path at a suitable sensor position. The sensor position can be determined by means of simulation and/or measurement and can be a position on the fan stator or on the electric motor at which a volumetric flow rate difference absolute value determined at the sensor position is less than a predefined limit value or at which the volumetric flow rate difference absolute value is minimum during a change of the volumetric flow rate by the fan in its operating range from a minimum volumetric flow rate to a maximum volumetric flow rate. Thus, the sensor position on the fan stator or on the electric motor can be determined at which a volumetric flow rate change by the fan has a minor or the least influence on the measurement value of the gas sensor. Due to the determination of this sensor position, the compensation based on correction data can be carried out with sufficient accuracy.

Any embodiment of the monitoring device described above can be set up or calibrated as follows:

First, a reference value for the quality of the gas atmosphere in a non-flowing gas atmosphere can be determined and stored. For example, the reference value can be determined by the gas sensor in the gas atmosphere during standstill of the fan. Subsequently, the fan can be operated in its operating range from a minimum volumetric flow rate to a maximum volumetric flow rate, wherein at multiple volumetric flow rate values, one calibration measurement value is detected respectively by the gas sensor. Subsequently, the following data can be used and stored as correction data:
the reference value and the determined value pairs comprising one calibration measurement value and the assigned volumetric flow rate respectively; or
determination of one correction value respectively based on a calibration measurement value and the reference value, wherein the value pairs of one correction value and the assigned volumetric flow rate respectively form the correction data.

In the latter case the correction values can be the difference between one calibration measurement value and the reference value respectively, for example.

The correction data can be described in a suitable form by means of a table, a function or a curve. If the correction values are not available for all of the volumetric flow rate values at which the fan can be operated, the correction data can be determined for the current volumetric flow rate value respectively from the available correction data, e.g. by means of interpolation or extrapolation or other suitable mathematic methods. If the volumetric flow rate of the fan can be varied only stepwise over its operating range, a value pair of a reference value and a volumetric flow rate value or of a correction value and a volumetric flow rate value can be provided in the correction data for each step.

With use of any embodiment of the monitoring device the quality of the gas atmosphere and particularly the air atmosphere can be monitored during operation of the fan of a room air system of which the fan is a component.

### BRIEF DESCRIPTION OF THE DRAWINGS

Advantageous configurations of the present disclosure are derived from the dependent claims, the description and the drawings. In the following, preferred embodiments of the present disclosure are explained in detail based on the enclosed drawings. The drawings show:

FIG. 1 a schematic block diagram of an embodiment of a monitoring device,

FIG. 2 a schematic block-diagram-like illustration of an embodiment of a fan of the monitoring device of FIG. 1 in the form of a radial fan,

FIG. 3 a schematic block-diagram-like illustration of an embodiment of a fan of the monitoring device of FIG. 1 in form of an axial fan,

FIG. 4 a perspective illustration of an embodiment of a monitoring device comprising a fan in form of a radial fan,

FIG. 5 a schematic exemplary progress of a reference value, of a calibration measurement value as well as a correction value depending on the volumetric flow rate of a gas flow in the gas atmosphere and

FIG. 6 a schematic illustration for determination of a volumetric flow rate independent monitoring value based on a measurement value characterizing the quality of the gas of the gas atmosphere, as well as a correction value depending on a current volumetric flow rate at which the measurement value has been measured.

### DETAILED DESCRIPTION

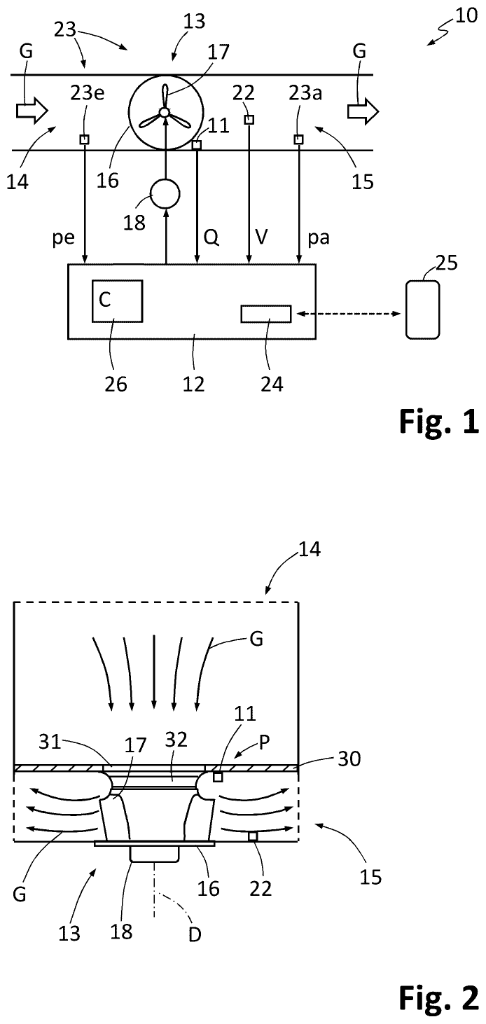

In FIG. 1 a block diagram of an embodiment of a monitoring device 10 is illustrated. The monitoring device 10 is configured to monitor the quality of a gas atmosphere A. The gas atmosphere A can be an air and particularly a room air atmosphere. For this purpose, the monitoring device 10 comprises a gas sensor 11 that creates a measurement value Q, which describes a parameter of the quality of the gas or gas mixture of the gas atmosphere A, e.g. the total concentration of volatile organic compounds (TVOC value) or the concentration of dust particles of a defined size, e.g. PM 2.5. The gas sensor 11 can also be configured to provide multiple different parameters or measurement values Q. The gas sensor 11 is communicatively connected to a control device 12 of the monitoring device 10 and provides the measurement value Q to the control device 12.

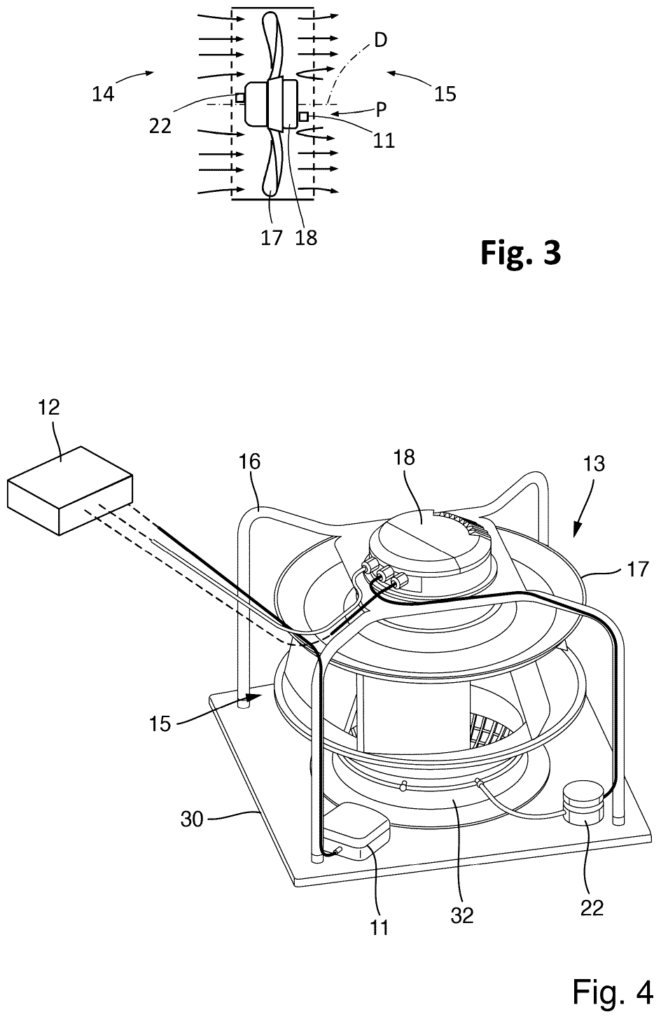

The monitoring device 10 comprises a fan 13 that is configured to produce a gas flow G having a variable volumetric flow rate V. The fan can be part of a room air system, which is present or which is to be installed. The fan has a suction side 14 on which it sucks gas out of the gas atmosphere A and a pressure side 15 on which it ejects the gas flow. The fan 13 can be configured as radial fan (FIGS. 2 and 4) or as axial fan (FIG. 3).

The flow direction in the main gas flow path of the gas flow G upstream and downstream of fan 13 depends on one hand from the type of the fan 13 (radial fan or axial fan) as well as from the configuration and the extension of flow channels of the room air system and can vary depending on the application. The predefined, desired flow direction of the gas flow G can be considered as main gas flow path, e.g. parallel or orthogonal to the rotation axis D, depending on how the type of the fan 13 (radial fan or axial fan) as well as the flow channels of the room air system adjoining a fan 13 define it as setpoint flow direction. In the block diagram of FIG. 1 only schematically and by way of example, a substantially straight main flow path of the gas flow G is illustrated.

The fan 13 has a fan stator 16 as well as a fan rotor 17. The fan rotor 17 is rotatingly supported around a rotation axis D on the fan stator 16. The fan rotor 17 is drivingly connected with an electric motor 18 of fan 13. For example, fan rotor 17 can be torque-proof connected with the rotor of the electric motor 18, so that the rotational speed of the electric motor 18 is equal to the rotational speed of the fan rotor 17 (fan rotational speed).

The electric motor 18 is controlled by control device 12. Basically, electric motor 18 can be any type of electric motor and can be configured as brushless DC-motor.

As for example schematically illustrated in FIG. 1, the monitoring device 10 can comprise one more additional sensors in addition to gas sensor 11, e.g. in order to detect additional parameters of the gas atmosphere A or the gas flow G, such as a temperature sensor, a humidity sensor, etc. According to the example, in addition a volumetric flow rate sensor 22 is arranged in a main flow path of gas flow G, preferably downstream of fan 13, which is configured to detect a respectively current value for the volumetric flow rate V of gas flow G and to provide it for control device 12. As additional option, a differential pressure sensor arrangement 23 can be provided in order to measure or determine a differential pressure dp between suction side 14 and pressure side 15 of fan 13. The differential pressure dp is the difference between an output pressure pa on the pressure side 15 and input pressure pe on the suction side 14 of fan 13. The differential pressure sensor arrangement 23 can comprise an input pressure sensor 23e for detection of the input pressure pe and an output pressure sensor 23a for detection of the output pressure pa, for example.

Also, the at least one optional sensor (e.g. volumetric flow rate sensor 22 and/or differential pressure sensor arrangement 23) present in addition to gas sensor 11 are communicatively connected to control device 12.

In modification to the measurement of the volumetric flow rate V by means of a volumetric flow rate sensor 22, the volumetric flow rate V can also be determined or calculated in the control device 12. For example, the fan rotational speed of fan 13 and the torque of the electric motor 18 can be used for determination of the volumetric flow rate V. The torque and the rotational speed can be assigned to a volumetric flow rate V by means of the fan characteristic curve, wherein the torque is characteristic for the differential pressure dp between suction side 14 and pressure side 15 of fan 13.

In the embodiment illustrated in FIG. 1 control device 12 can comprise a communication module 24 that is configured to establish a communication connection to an external device 25, e.g. a mobile device, such as a smartphone, a tablet computer, a notebook, etc. The communication connection is preferably wireless and according to the example is based on a defined communication protocol, e.g. Bluetooth, Bluetooth Low Energy (BLE), ZigBee, WiFi, etc. Additionally or alternatively, the communication connection between the communication module 24 and the external device 25 can also be established via a Wireless Local Area Network (WLAN) and/or the mobile phone network.

According to the example, control device 12 comprises in addition a non-volatile memory 26 in which data can be stored, e.g. in order to set up and/or calibrate monitoring device 10. Additionally or alternatively, the control device 12 can be communicatively connected to a non-volatile external memory (e.g. Cloud-memory) in order to secure data in redundant manner in addition to the local storage and/or to provide data for download. The set-up of the monitoring device 10 can be carried out prior to or during the first start-up as factory setting and/or at the installation site. The set-up of the monitoring device 10 can be carried out based on the flow conditions of gas flow G of fan 13 solely or in additional consideration of the flow conditions of gas flow G within the room air system in which the fan is installed or shall be installed at the installation site.

In the context of set-up of monitoring device 10, first the sensor position P of gas sensor 11 is determined by means of measurement and/or simulation. The gas sensor 11 is arranged on the fan stator 16 or on a non-rotating component of electric motor 18, such as a motor housing or the stator of the electric motor. The sensor position P is thereby selected so that the varying volumetric flow rate V of gas flow G influences the detected measurement value Q of gas sensor 11 as little as possible. If gas sensor 11 measures, for example, the concentration of volatile components or dust particles in the gas atmosphere A, the detected measurement value Q depends on the current value of the volumetric flow rate V created by fan 13. Therefore, the sensor position P is selected outside of the main flow path of gas flow G.

A position can be selected as sensor position P based on a measurement or a simulation at which the flow conditions change less than in the main flow path and particularly as little as possible if fan 13 sets the volumetric flow rate V variably over its operating range between a minimum volumetric flow rate and a maximum volumetric flow rate. For example, the flow can be measured or simulated in the area of fan 13 with or without consideration of the flow channels of the room air system if the volumetric flow rate V is varied between the minimum and maximum volumetric flow rate V or at least over an extensive part of more than 50%-70% of the entire operating range. Therefrom possible sensor positions can be recognized at which the flow conditions change remarkably less than in the main flow path of the gas flow.

In the illustrated embodiments the sensor position is at a position downstream of fan rotor 17. For example, the sensor position P can be offset relative to the fan rotor 17 and particularly the fan blades of fan rotor 17 obliquely or orthogonal to the flow direction of gas flow G inside the main flow path.

In the radial fan, according to FIGS. 2 and 4, the sensor position P can be arranged on the backside of a front plate of fan stator 16, through which the radial fan sucks gas through an inlet 31 inside front plate 30 for creation of the gas flow. The inlet 31 is enclosed by a ring 32 of fan stator 16 adjoining the front plate 30. The gas of gas flow G flows from inlet 31 through ring 32 and further toward fan rotor 17 or the fan blades or fan rotors 17. The gas sensor 11 is arranged next to the ring 32 according to the example. It is arranged offset from fan rotor 17 transverse to the flow direction of gas flow G.

In the axial fan schematically illustrated in FIG. 3, gas sensor 11 can be arranged on a surface facing away from gas flow G in the proximity of rotation axis D, e.g. on a housing of the electric motor 18.

After the sensor position P has been defined based on a measurement and/or simulation, the monitoring device 10 is calibrated, whereby it is proceeded as follows:

First, a reference value R is measured in the gas atmosphere A with a gas movement as low as possible, e.g. during standstill of fan 13, e.g. by means of the gas sensor 11. The reference value R describes the same parameter as measurement value Q, e.g. the concentration of dust particles having a defined size in the gas atmosphere A or the concentration of volatile organic compounds (e.g. TVOC value).

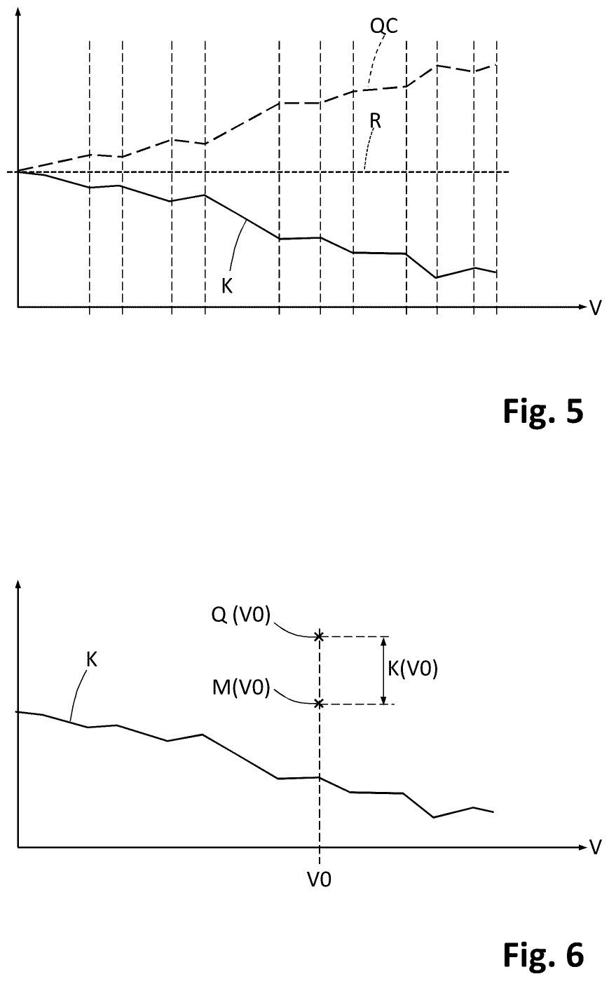

Subsequently, fan 13 is operated with varying volumetric flow rate V. In the operating range of fan 13 from a minimum volumetric flow rate value to a maximum volumetric flow rate value multiple measurement values Q are measured for one assigned volumetric flow rate value respectively, which can be denoted as calibration measurement values QC. In doing so, value pairs of respectively one calibration measurement value QC and the assigned value of the volumetric flow rate V at which the calibration measurement value QC has been detected, are created. A schematic only exemplary curve of such value pairs for the calibration measurement values QC is illustrated in FIG. 5. There it is apparent that the deviation between the calibration measurement values QC and the reference value R increases with increasing volumetric flow rate V.

Based on reference value R and the calibration measurement values QC, volumetric flow rate dependent correction values K can be optionally calculated, e.g. due to calculation of the difference between the calibration measurement values QC and the reference value R:

$$K ( V ) = QC ( V ) - R .$$

Based on the measurement of the calibration measurement values QC, correction data C can be stored in the memory 26 of control device 12. The following can be used as correction data C:
(a) the reference value R and the volumetric flow rate dependent calibration measurement values QC and/or
(b) the volumetric flow rate dependent correction values K.

The correction data C can be stored in suitable manner, e.g. by storing the reference value R and the value pairs of respectively one calibration measurement value QC and the assigned value of the volumetric flow rate V (compare option (a)) and/or by storing the value pairs of respectively one correction value K and the assigned value of the volumetric flow rate V (compare option (b)). The correction data C can be stored by means of a table, a curve, an approximated function, etc. Thereby it is possible by means of interpolation, extrapolation or other suitable mathematic methods to determine intermediate values for correction data C, which have not been determined by means of measurement or simulation.

Based on correction data C, the current value for the volumetric flow rate V, which can be determined by means of the volumetric flow rate sensor 22, for example during the operation of the monitoring device 10, a volumetric flow rate independent monitoring value M can be determined in the control device 12. The monitoring value M is based on the current measurement value Q, the current for the volumetric flow rate V as well as the correction value K associated to the current volumetric flow rate as follows:

$$M ( V ) = Q ( V ) - K ( V ) .$$

The method is schematically illustrated in FIG. 6. The current measurement value Q is corrected based on the correction value K assigned to the current value for the volumetric flow rate V, so that the obtained monitoring value M is corrected and is thus, at least substantially independent from the current volumetric flow rate V created by fan 13.

By means of the present disclosure it is possible to correct measurement values Q detected by gas sensor 11 during operation of the monitoring device 10, so that the use with fans 13 is made possible, which create a varying volumetric flow rate V during operation. Additional fans associated to the gas sensor 11, which permanently produce the same constant volumetric flow rate, can be omitted. The monitoring device 10 can be realized with fans 13 in a very simple and economic manner, which are or shall be installed in a room air system.

The present disclosure refers to a monitoring device 10 and a method for monitoring the quality of a gas atmosphere A. The monitoring device 10 comprises a fan 13 configured to produce a gas flow G having a variable volumetric flow rate V. A gas sensor 11 is arranged at a sensor position P on an immovable part of fan 13 and provides a measurement value Q for a control device 12. The control device 12 is configured to determine a volumetric flow rate corrected and at least substantially volumetric flow rate independent monitoring value M based on the measurement value Q and provided correction data C.

### LIST OF REFERENCE SIGNS

10 monitoring device
11 gas sensor
12 control device
13 fan
14 suction side
15 pressure side
16 fan stator
17 fan rotor
18 electric motor
22 volumetric flow rate sensor
23 differential pressure sensor arrangement
23a output pressure sensor
23 input pressure sensor
24 communication module
25 external device
26 memory
30 front plate
31 inlet
32 ring
A gas atmosphere
C correction data
D rotation axis
dp differential pressure
G gas flow
K correction value
M monitoring value
P sensor position
pa output pressure
pe input pressure
Q measurement value
QC calibration measurement value
R reference value
V volumetric flow rate

1. A monitoring device configured for monitoring the quality of a gas atmosphere, comprising:
a fan configured to create a gas flow having a variable volumetric flow rate and comprising a fan rotor and an electric motor, wherein the fan rotor is rotatingly supported around a rotation axis on a fan stator,
a gas sensor arranged on the fan stator or on the electric motor and being configured to produce a measurement value characterizing the quality of a gas of the gas atmosphere,
a control device, which is communicatively connected to the gas sensor and which determines a volumetric flow rate corrected monitoring value based on the measurement value, a current volumetric flow rate and correction data.

2. The monitoring device according to claim 1, further comprising a volumetric flow rate sensor for measurement of the current volumetric flow rate, which is communicatively connected to the control device.

3. The monitoring device according to claim 1, wherein the control device is configured to determine the current volumetric flow rate based on at least one operating parameter of the fan and/or the electric motor.

4. The monitoring device according to claim 1, wherein the control device is configured to determine the current volumetric flow rate based on a rotational speed and a torque of the electric motor or based on operating parameters correlated therewith.

5. The monitoring device according to claim 1, further comprising a differential pressure sensor arrangement for measurement of a differential pressure between a suction side and a pressure side of the fan, wherein the control device is configured to control the differential pressure in open loop or closed loop manner.

6. The monitoring device according to claim 1, wherein the gas sensor is arranged outside a main gas flow path of the gas flow.

7. The monitoring device according to claim 1, wherein the gas sensor is arranged at a sensor position at which a determined volumetric flow rate difference absolute value is below a predefined limit value during a variation of a volumetric flow rate created by the fan from a minimum volumetric flow rate to a maximum volumetric flow rate.

8. The monitoring device according to claim 1, wherein the gas sensor is arranged at a sensor position at which a determined volumetric flow rate difference absolute value is least during a variation of a volumetric flow rate created by the fan from a minimum volumetric flow rate to a maximum volumetric flow rate.

9. A method for set-up of a monitoring device for monitoring the quality of a gas atmosphere, wherein the monitoring device comprises a fan for producing a gas flow having a variable volumetric flow rate having a fan rotor and an electric motor, wherein the fan rotor is rotatingly supported around a rotation axis on a fan stator, a gas sensor arranged on the fan stator or on the electric motor and a control device communicatively connected to the gas sensor, wherein the method comprises the following steps:
determining a reference value for the quality of a gas of the gas atmosphere,
operating the fan so that a volumetric flow rate varies from a minimum volumetric flow rate to a maximum volumetric flow rate and creating a calibration measurement value at different volumetric flow rates respectively, which characterize the quality of a gas of the gas atmosphere,
storing the reference value and value pairs consisting of one calibration measurement value and an assigned volumetric flow rate respectively as correction data or determining one correction value respectively from the reference value and one of calibration measurement values and storing the value pairs consisting of one correction value and the assigned volumetric flow rate respectively as correction data.

10. The method according to claim 9, wherein a sensor position, at which the gas sensor is arranged, is determined by means of measurement or simulation of the gas flow, wherein a position on the fan stator or on the electric motor is selected as sensor position at which a determined volumetric flow rate difference absolute value is below a predefined limit value during a variation of the volumetric flow rate created by the fan from a minimum volumetric flow rate to a maximum volumetric flow rate.

11. A method for monitoring the quality of a gas atmosphere using a monitoring device, comprising a fan for producing a gas flow having a variable volumetric flow rate having a fan rotor and an electric motor, wherein the fan rotor is rotatingly supported around a rotation axis on a fan stator, a gas sensor arranged on the fan stator or the electric motor and a control device communicatively connected to the gas sensor, wherein the method comprises the following steps:
detection of a measurement value that characterizes the quality of a gas of the gas atmosphere by means of the gas sensor,
determining a volumetric flow rate corrected monitoring value based on the measurement value, a current volumetric flow rate and predefined correction data.

12. The monitoring device according to claim 2, further comprising a differential pressure sensor arrangement for measurement of a differential pressure between a suction side and a pressure side of the fan, wherein the control device is configured to control the differential pressure in open loop or closed loop manner.

13. The monitoring device according to claim 12, wherein the gas sensor is arranged outside a main gas flow path of the gas flow.

14. The monitoring device according to claim 13, wherein the gas sensor is arranged at a sensor position at which a determined volumetric flow rate difference absolute value is below a predefined limit value during a variation of a volumetric flow rate created by the fan from a minimum volumetric flow rate to a maximum volumetric flow rate.

15. The monitoring device according to claim 14, wherein the gas sensor is arranged at a sensor position at which a determined volumetric flow rate difference absolute value is least during a variation of a volumetric flow rate created by the fan from a minimum volumetric flow rate to a maximum volumetric flow rate.

16. The monitoring device according to claim 3, further comprising a differential pressure sensor arrangement for measurement of a differential pressure between a suction side and a pressure side of the fan, wherein the control device is configured to control the differential pressure in open loop or closed loop manner.

17. The monitoring device according to claim 16, wherein the gas sensor is arranged outside a main gas flow path of the gas flow.

18. The monitoring device according to claim 17, wherein the gas sensor is arranged at a sensor position at which a determined volumetric flow rate difference absolute value is below a predefined limit value during a variation of a volumetric flow rate created by the fan from a minimum volumetric flow rate to a maximum volumetric flow rate.

19. The monitoring device according to claim 18, wherein the gas sensor is arranged at a sensor position at which a determined volumetric flow rate difference absolute value is least during a variation of a volumetric flow rate created by the fan from a minimum volumetric flow rate to a maximum volumetric flow rate.

20. The monitoring device according to claim 4, further comprising a differential pressure sensor arrangement for measurement of a differential pressure between a suction side and a pressure side of the fan, wherein the control device is configured to control the differential pressure in open loop or closed loop manner.
